(19) United States
(12) Patent Application Publication
(10) Pub. No.: US 2006/0057592 A1
(43) Pub. Date: Mar. 16, 2006

(54) Methods for identifying primase trinucleotide initiation sites and identification of inhibitors of primase activity

(76) Inventors: Mark Griep, Lincola, NE (US); Steven Hinrichs, Omaha, NE (US); Scott Koepsell, Mission Hill, SD (US); Khalid Sayood, Lincoln, NE (US); James M. Takacs, Lincoln, NE (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/941,717

(22) Filed: Sep. 15, 2004

(51) Int. Cl. C12Q 1/68

(52) U.S. Cl. 435/6

(57) ABSTRACT

Methods and kits for the identification of a primase trinucleotide initiation site and for the identification of compounds which modulate bacterial primase activity are provided.

### FIELD OF THE INVENTION

The present invention relates to the modulation of bacterial primase activity and to methods for the identification of new antibiotics which target bacterial primase.

### BACKGROUND OF THE INVENTION

Several publications and patent documents are cited throughout the specification in order to describe the state of the art to which this invention pertains. Each of these references is incorporated herein by reference as though set forth in full.

Primase is a DNA-dependent RNA polymerase that functions at the replication fork on single-stranded DNA (ssDNA) to create primers de novo for elongation of both leading- and lagging-strand DNA polymerases (Frick and Richardson (2001) Annu. Rev. Biochem. 70:39-80; Griep, M. A., Primase Entry, in: S. Brenner, J. Miller (Eds.), Encyclopedia of Genetics, Academic Press, New York, 2001, pp. 1542-1545). All known DNA polymerases require a C-3′-hydroxyl group to initiate nucleotide polymerization, whereas primase is uniquely capable of de novo synthesis. Bacteria with conditionally lethal primase mutations lack the ability to replicate chromosomal DNA under the restrictive conditions (Grompe, M., et al. (1991) J. Bacteriol. 173:1268-1278). Prokaryotic primases significantly differ in their structure from eukaryotic primases despite performing the same function (Augustin, M. A., et al. (2001) Nat. Struct. Biol. 8:57-61; Griep, M. A. (1995) Indian J. Biochem. Biophys. 32:171-178). Since primase is an essential protein for replication, it has been identified as a potential target for new antibiotic drug development, especially considering that the potential exists to generate selective inhibitors of prokaryotic primases over eukaryotic primase.

Escherichia coli primase specifically recognizes the trinucleotide d(CTG) sequence, initiates primer synthesis complementary to the thymine, and proceeds in the 5′ direction of the template (Bhattacharyya and Griep (2000) Biochemistry 39:745-752). The cryptic guanine is required for primase to initiate primer synthesis, but its complement is not incorporated into the de novo primer. In addition to de novo primer synthesis, primase is able to elongate primed ssDNA, creating a newly synthesized complementary RNA strand (Johnson, S. K., et al. (2000) Biochemistry 39:736-744). This process appears to occur on a ssDNA template that forms a 3′ hairpin structure, yielding an RNA-DNA copolymer termed an “overlong primer.”

To date, assays for measuring primase activity have monitored the incorporation of radiolabeled nucleotides into the growing primer. Variations include a recently developed high-throughput assay that measures primase activity but does not provide qualitative information on the nature of the primers synthesized (Zhang, Y., et al. (2002) Anal. Biochem. 304:174-179). Such qualitative information provides potentially valuable data for characterizing how an inhibitor functions. Other assays have electrophoretically separated the radiolabeled primers followed by autoradiography to visualize them (Swart and Griep (1995) Biochemistry 34:16097-16106; Swart and Griep (1993) J. Biol. Chem. 268:12970-12976). While yielding sensitivity and RNA primer information such as yield and size, these assays are relatively time consuming and provide information only about primers that have incorporated the radiolabeled nucleotide.

### SUMMARY OF THE INVENTION

In accordance with the present invention, methods are provided for identifying the initiation sequence of a bacterial primase. In a particular embodiment, the method comprises the steps of: contacting the bacterial primase with a template nucleic acid molecule comprising a candidate initiation sequence; placing the mixture comprising the primase and template nucleic acid molecule under conditions which promote primase activity; and identifying the reaction products. The reaction products can be identified by any method including, without limitation, monitoring incorporation of radiolabeled nucleotides and performing thermally denaturing high performance liquid chromatography (DHPLC). In a preferred embodiment, the reaction products are detected by DHPLC. The presence of a nucleic acid molecule, specifically a primer, other than the template nucleic acid molecule indicates that the candidate initiation sequence is an initiation sequence recognized by the bacterial primase. Preferably, the template nucleic acid molecule is single-stranded DNA. In a particular embodiment of the invention, the single-stranded DNA is blocked at the 3′ end.

According to another aspect of the instant invention, the candidate initiation sequence is identified by searching for trinucleotides present in the bacterial genome at a high clustering frequency. In a preferred method, the size of the window searched and the threshold are accounted for in determining the clustering frequency.

In another embodiment of the invention, methods for identifying inhibitors of bacterial primase activity are provided. In a particular embodiment of the invention, the method comprises the steps of: 1) contacting the bacterial primase with a template nucleic acid molecule comprising its initiation sequence and a compound suspected of possessing primase inhibiting activity; 2) placing the mixture comprising primase, template nucleic acid, and candidate compound under reaction conditions suitable for primase activity; and 3) quantitating the reaction products, such as by DHPLC. The detection of reduced amounts of a nucleic acid molecule (i.e., an RNA primer), other than the template nucleic acid molecule, in the presence of the candidate compound, indicates that the candidate compound inhibits bacterial primase activity.

In accordance with yet another aspect of the instant invention, additional methods for identifying a compound which inhibits bacterial primase activity are provided. In a particular embodiment, the method comprises the steps of: 1) obtaining a computer model of the zinc-binding domain of the bacterial primase; 2) identifying amino acids, preferably surface amino acids, of the bacterial primase which are heterologous to the corresponding amino acids of at least one other bacterial primase which recognizes a trinucleotide initiation site different than the initiation site recognized by said bacterial primase; 3) and identifying the binding sites of a candidate compound. The overlap of the binding site of a candidate compound with the identified heterologous amino acids indicates the candidate compound likely inhibits bacterial primase activity. In a particular embodiment of the instant invention, the heterologous amino acids determine the initiation specificity of the bacterial primer. According to yet another aspect of the invention, the ability of the heterologous amino acids to determine the initiation site specificity of the bacterial primase is determined by site-directed mutagenesis. Furthermore, the ability of the identified candidate compounds to inhibit primase activity can be measured by the methods described hereinabove or by administration of the compound to the bacteria, wherein the inhibition of bacterial growth indicates the candidate compound inhibits primase activity.

In yet another embodiment of the instant invention, kits are provided for performing the methods of the instant invention. In a particular embodiment, the kits include 1) a set of single-stranded DNA molecules, each with a different trinucleotide sequence composed of G, A, C, and T nucleotides and each being capable of binding a bacterial primase, 2) a primase buffer, 3) ribonucleoside triphosphates (rNTPs), and 4) a magnesium salt. The kits may also optionally include at least one of: an HPLC column, wash buffers, elution buffers, and instruction material.

In accordance with another aspect of the instant invention, compounds are provided which inhibit bacterial primase activity.

### BRIEF DESCRIPTION OF THE DRAWING

FIG. 1 contains chromatograms of oligonucleotides eluted from thermally denaturing high performance liquid chromatography (DHPLC). The oligonucleotides were incubated in the presence or absence of primase prior to RP-HPLC analysis. Arrows indicate primer RNA.

FIG. 2 contains reaction schemes for de novo primer synthesis (FIG. 2A) and elongation from 3′-hairpins (FIG. 2B). In the presence of all four rNTPs, an RNA primer is synthesized complementary to its recognition sequence 5′-CTG-3′ (boldface) when the 3′-hydroxyl group on the template is blocked by a C3 linker (FIG. 2A) or other blocking agents. In the absence of a C3 linker, a similar template can form a 3′-hairpin, allowing primase to elongate from the exposed 3′-hydroxyl group (FIG. 2B).

FIG. 3 contains chromatograms of thermally denaturing HPLC (DHPLC) analysis of de novo primase activity (FIG. 3A) and elongation from a 3′-hairpin (FIG. 3B). In FIG. 3A, the template-length-dependent RNA primers (enlarged inset) eluted before the ssDNA template (filled arrow). The major RNA peak (open arrow) eluted at 8.49 minutes. In FIG. 3B, the overlong primers (open arrow) eluted before the ssDNA template (filled arrow). The reactions were performed without primase (i) or with primase for 1 hour (ii), 2 hours (iii), or 4 hours (iv). Reactions were performed as described hereinbelow and analyzed by DHPLC at 80° C. with a 0-8.1% acetonitrile gradient over 16 minutes for de novo primers and a 8.5-12% acetonitrile gradient over 8 minutes for overlong primers.

FIG. 4 is a graph of the elution of oligonucleotides from DHPLC analysis of single-stranded DNA (triangles), RNA (circles), and uracil-containing DNA (squares). The sequences employed were 12-mer, 16-mer, and 18-mer. The oligonucleotides were analyzed under the same conditions used for de novo primers.

FIG. 5 demonstrates sequence-specific insertion of a nucleotide into a primer. De novo primer synthesis using the ssDNA template 5′-CAGA(CA)₅CTG(CA)₃-C3-3′ was carried out in the absence of rCTP (trace A (FIG. 5A), lane 3 (FIG. 5B)), or in the presence of 5 μM ddCTP (trace B (FIG. 5A), lane 4 (FIG. 5B)). Reactions were performed as described hereinbelow. 8 μl was analyzed by HPLC (FIG. 5A) and 3 μl was analyzed by polyacrylamide gel electrophoresis (FIG. 5B).

FIG. 6 is a graph depicting de novo primer synthesis kinetics of the 16-mer template-length-dependent RNA primer. Known amounts of the control RNA 16-mer 5′-AG(UG)₇-3′ were used to generate a standard curve that was used to convert the peak area of the major RNA primer peak into picomoles. Reactions were performed for the indicated amounts of time as described hereinbelow. The data were fitted with a curve as described with a Yₘₐₓ of 3.96 pmol and a rate constant of 0.00251 s¹. Each data point is the average of three experiments, and the error bars show the standard error.

FIG. 7 is a graph depicting the inhibition of de novo primer synthesis by a mixture of four dNTPs. Primer synthesis was carried out as described hereinbelow for 1 hour in the presence of the indicated amounts of dNTPs. Total primer area in the absence of dNTPs was set to 100% activity. The curve was fitted with an IC50 of 9.5 μM.

FIG. 8 contains chromatograms of oligonucleotides from DHPLC analysis. The oligonucleotides were incubated in the presence (top) or absence (bottom) of primase prior to DHPLC analysis.

FIGS. 9A-9C represent clustering of trinucleotide sequences in E. coli (FIG. 9A), B. anthracis (FIG. 9B), and Y. pestis (FIG. 9C). Each panel represents the relative clustering of a particular trinucleotide as a function of window size and threshold. Low clustering shows up as black while higher levels of clustering are represented by shades of gray.

### DETAILED DESCRIPTION OF THE INVENTION

Modern approaches to the design of new antibiotics are based on molecular biology techniques requiring knowledge of the structure and function of the target. The instant invention relates to methods for elucidating the key elements of a new target for antibiotic development. Specifically, methods for identifying inhibitors of the bacterial enzyme primase are provided. Identified inhibitors of bacterial primase can be employed to inhibit the growth of the bacteria.

The structure of a key primase element relevant to the instant invention is the amino-terminal zinc-binding domain (ZBD), which is typically about 110 residues. Its structure had been previously determined from the primase gene of B. stearothermophilus. Bacterial (DnaG) primase is thought to be an excellent target for new antimicrobial drug development because 1) it differs from the primase of the eukaryotes, e.g., humans; 2) it plays an essential role in cellular replication; and 3) resistance mechanisms are not known to exist. Bacterial primases are very interesting in that they have the ability to initiate primer synthesis in a very specific manner. The three nucleotides recognized by a given bacterial primase are believed to be unique. For example, E. coli primase binds to CTG but it is expected that other bacteria will bind to other trinucleotides sequences such as TTA. Notably, the specificity-determining region may be unique to an entire genus or several genera such that a single inhibitory compound may be effective against a variety of bacteria. Alternatively, the specificity determining region may be unique to a single species or a limited number of species such that an inhibitory compound would be effective against a narrow subset of bacteria.

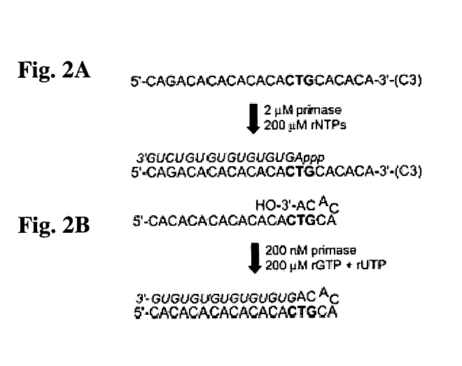

Additionally, the instant invention provides an automated, scalable, and rapid HPLC assay to assess primase activity without the cost, safety, and time issues associated with radioactivity. The new HPLC assay yields quantitative information on the nature of the primers synthesized and can be completed in less time than electrophoretic assays, such as those employed to detect radiolabeled nucleotides. The HPLC assay uses a synthetic ssDNA template that incorporates two essential features required for de novo primase activity, including the primase recognition sequence 5′-d(CTG)-3′ and six nucleotides 3′ to the initiation sequence believed to be necessary for the structural support that primase needs to bind ssDNA (FIG. 2A; Yoda and Okazaki (1991) Mol. Gen. Genet. 227:1-8).

The primases of the instant invention can be from any bacteria. The bacteria can be from any genus including, without limitation, Staphylococci (e.g., S. aureus), Streptococci (e.g., S. pneumoniae), Clostridia (e.g., C. perfringens, C. tetani), Neisseria (e.g., N. gonorrhoea), Enterobacteriaceae (e.g., E. coli), Helicobacter (e.g., H. pylori), Vibrio (e.g., V. cholerae), Capylobacter (e.g., C. jejuni), Pseudomonas (e.g., P. aeruginosa), Haemophilus (e.g., H. influenzae), Bordetella (e.g., B. pertussis), Mycoplasma (e.g., M. pneumoniae), Ureaplasma (e.g., U. urealyticum), Legionella (e.g., L. pneumophila), Treponema, Leptospira, Borrelia (e.g., B. burgdorferi), Mycobacteria (e.g., M. tuberculosis, M. smegmatis), Listeria (e.g., L. monocytogenes), Actinomiyces (e.g., A. israelii), Nocardia (e.g., N. asteroides), Chlamydia (e.g., C. trachomatis), Rickettsia, Coxiella, Rochalimaea, Brucella, Yersinia (e.g., Y. pestis), Francisella (e.g., F. tularensis), Bacillus (e.g., B. anthracis, B. subtilis, B. stearothermophilus), and Pasteurella. In a particular embodiment of the invention, the bacteria is selected from the group consisting of: F. tularensis, S. aureus, B. anthracis, H. pylori, M. tuberculosis, and Y. pestis. In another embodiment, the bacteria is F. tularensis.

I. Definitions

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An “isolated nucleic acid” (either DNA or RNA) may further represent a molecule produced directly by biological or synthetic means and separated from other components present during its production.

The terms “percent similarity”, “percent identity” and “percent homology” when referring to a particular sequence are used as set forth in the University of Wisconsin GCG software program.

The term “substantially pure” refers to a preparation comprising at least 50-60% by weight of a given material (e.g., nucleic acid, oligonucleotide, protein, etc.). More preferably, the preparation comprises at least 75% by weight, and most preferably 90-95% by weight of the given compound. Purity is measured by methods appropriate for the given compound (e.g. chromatographic methods, agarose or polyacrylamide gel electrophoresis, HPLC analysis, and the like).

The term “oligonucleotide” as used herein refers to sequences, primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The term “primer” as used herein refers to an oligonucleotide, either RNA or DNA, either single-stranded or double-stranded, either derived from a biological system, generated by restriction enzyme digestion, generated by an enzyme such as primase, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as appropriate temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos. 4,683,195, 4,800,195, and 4,965,188, the entire disclosures of which are incorporated by reference herein.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA molecule of the invention, to the substantial exclusion of hybridization of the oligonucleotide with single-stranded nucleic acids of non-complementary sequence. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (Sambrook et al., 1989, Molecular Cloning: A Laboratory Manual, Cold Spring Harbor Laboratory Press):
Tₘ=81.5° C.+16.6 Log[Na+]+0.41(% G+C)−0.63(% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5× Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5× Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5× Denhardt's solution, 0.5% SDS and 100 μg/ml denatured salmon sperm DNA at 42° C., and washed in 0.1×SSC and 0.5% SDS at 65° C. for 15 minutes.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, or the addition of stabilizers.

The term “gene” refers to a nucleic acid comprising an open reading frame encoding a polypeptide, including both exon and (optionally) intron sequences. The nucleic acid may also optionally include non-coding sequences such as promoter or enhancer sequences. The term “intron” refers to a DNA sequence present in a given gene that is not translated into protein and is generally found between exons.

As used herein, “primase activity” refers to any activity normally associated with a primase, such as, without limitation, 1) the ability to synthesize a complementary RNA strand by elongation of a primed single-stranded DNA and 2) the ability to synthesize an RNA primer de novo.

II. Thermally Denaturing High Performance Liquid Chromatography

The thermally denaturing HPLC (DHPLC) of the instant invention is performed at elevated temperatures. Preferably, DHPLC is performed at a temperature high enough to dissociate an RNA and DNA complex. In a preferred embodiment, DHPLC is performed between 25° C. and 100° C. In a preferred method, DHPLC is performed at 80° C. Additionally, DHPLC may be performed using HPLC columns designed to separate nucleic acids. For example, DHPLC may be performed on alkylated nonporous polystyrene-divinylbenzene (PS-DVB) copolymer microsphere columns, such as the DNASep® reverse-phase column (Transgenomic; Omaha, Nebr.). General HPLC techniques are described in Ausubel et al., eds. (Current Protocols in Molecular Biology, John Wiley and Sons, Inc., (1995)).

III. Kits

The present invention also encompasses kits for use in performing the methods of the instant invention such as determining the initiation sequence of a bacterial primase, screening for compounds which modulate bacterial primase activity, and identifying compounds which modulate bacterial primase activity. Such kits include: 1) a set of single-stranded DNA molecules, each with a different trinucleotide sequence composed of G, A, C, and T nucleotides and each being capable of binding a bacterial primase, 2) a primase buffer, 3) ribonucleoside triphosphates (rNTPs), and 4) a magnesium salt. The kits may also optionally include at least one of: an HPLC column, wash buffers and elution buffers for performing HPLC, and instruction material.

As used herein, an “instructional material” includes a publication, a recording, a diagram, or any other medium of expression which can be used to communicate the usefulness of the composition of the invention for performing a method of the invention.

As used herein, a “primase buffer” is a buffer which does not inhibit and preferably promotes primase activity. The magnesium salt included in the kit can be, for example, magnesium acetate. Optionally, at least one of magnesium salt and the rNTPs may be included in the primase buffer.

IV. Rationally Designed Inhibitor

In a first approach, a series of compounds can be synthesized so that each compound: 1) has a backbone which fills a pocket or region of the primase ZBD (e.g., Pocket 3 of F. tularensis described hereinbelow), 2) has one group that binds strongly to the zinc, and 3) has other groups that give the inhibitor binding specificity. For example, Pocket 3 of F. tularensis was chosen because it lies between the ligated zinc and the initiation specificity residues that are unique to F. tularensis primase. For initial studies, a peptide mimetic of the initiation trinucleotide (e.g., d(TAT) of F. tularensis) can be generated (see, for example Formula I). 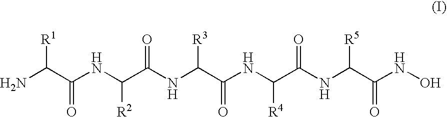
The peptide will include: 1) a polypeptide backbone, 2) a zinc-binding motif, exemplified here by hydroxamic acid, and 3) substituents, R¹-R⁵, to mimic the trinucleotide bases and give the inhibitor binding specificity. Specifically, substituents R¹-R³ may be designed to mimic the initiation trinucleotides and substituents R⁴ and R⁵ may be hydrogen or may be substituents which increase the binding specificity of the compound for the bacterial primase. Nucleotide mimics (e.g., analogs) are described hereinbelow.

Also provided hereinbelow is an exemplary compound, Tyr-Trp-Tyr-Glu-glycinehydroxamine acid (II). The compound includes: 1) tyrosines as substitutes for the thymines, 2) tryptophan as a substitute for the adenine, 3) an acidic residue at the third or fourth position to allow cyclization with the amino terminus, 4) a series of glycine linkers, and 5) glycinehydroxamic acid at its terminus. 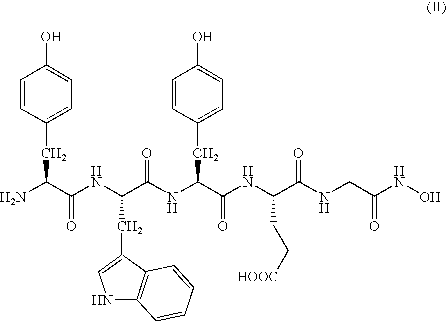
Notably, the L conformation of the compound is shown. However, D conformations are also contemplated as they are metabolized at a slower rate and therefore may prove to be more efficacious inhibitors in in vivo contexts. Additionally, the peptide backbone may be modified and the length of the —OH tail can be varied to maximize the fit of the compound for the primase (e.g., Pocket 3 of F. tularensis).

The tyrosines and tryptophans of II will give binding specificity and the hydroxamic acid will give binding affinity. The hydroxamic acid group binds strongly to zinc. In fact, there are several hydroxamic acid based metalloproteinase inhibitors that are currently in clinical trials. The E. coli primase zinc is very accessible to solvent even though it is ligated by three cysteines and one histidine. Additionally, it has been determined that zinc normally binds a fifth ligand when the enzyme binds substrates.

A second approach can be the use of non-peptide scaffolds to synthesize potential inhibitors via a combinatorial chemistry approach (see, for example, Formula III). The three side chain substituents, R¹-R³, will be designed to replace the nucleotide bases of the initiation trinucleotide (for example, two thymines bases and one adenine base for F. tularensis, as exemplified below). A zinc-binding motif, a hydrozamic acid or other group, is incorporated at a terminus of the scaffold.

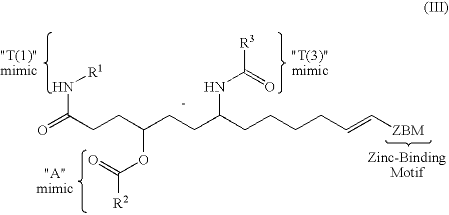

Zinc-binding motifs include, without limitation, ketones, diketones, ketoaldehydes, and carboxylates. Specific examples of zinc-binding motifs include, without limitation, hydrozamic acid, —CO₂H, —PO₃H₂, 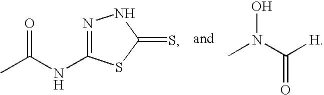
Nucleotide Mimics or Analogs are known in the art and include the following, without limitation: 1) thymine and cytosine can be mimicked by tyrosine, phenyl, pyridine, pyrimidine, and triazole moieties and derivatives thereof (e.g., 5-fluorouracil and 5-azacytidine) and 2) adenine and guanine can be mimicked by tryptophan, indole, and purine moieties and derivatives thereof (e.g., 6-mercaptopurine, 6-thioguanine, and 2-chloroadenine). Derivatives include moieties that are substituted with substituents including, without limitation, halo (e.g., F, Cl, Br, I); haloalkyl (e.g., CCl₃, CF₃), alkoxy (—OR); alkylthio (—SR); hydroxy (—OH); carboxy (—COOH); alkyloxycarbonyl (—C(O)R); alkylcarbonyloxy (—OCOR); amino (—NH₂); carbamoyl (—NHCOOR—, —OCONHR—); urea (—NHCONHR—); thiol (—SH); and alkyl (an optionally substituted straight, branched or cyclic hydrocarbon group, optionally saturated, preferably having from about 1-6 carbons), wherein R is an alkyl. Specific non-limiting examples of pyrimidine (thymine and cytosine) mimics include the following (shown as R¹NH₂): 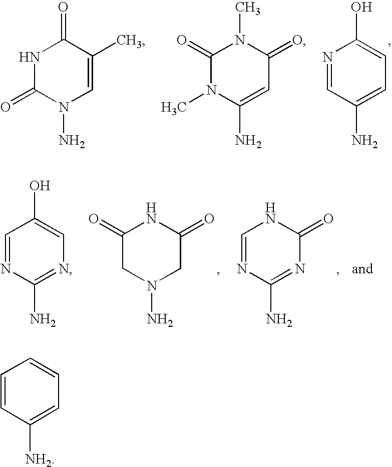
Additional examples of pyrimidine mimics include, without limitation, the following (shown as R³CO₂H): 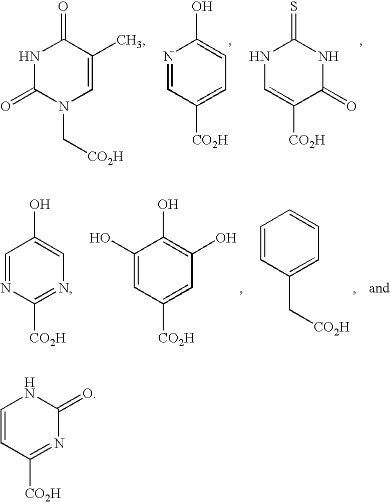
Specific examples of purine (adenoside and guanine) mimics include, without limitation (shown as R²CO₂H):

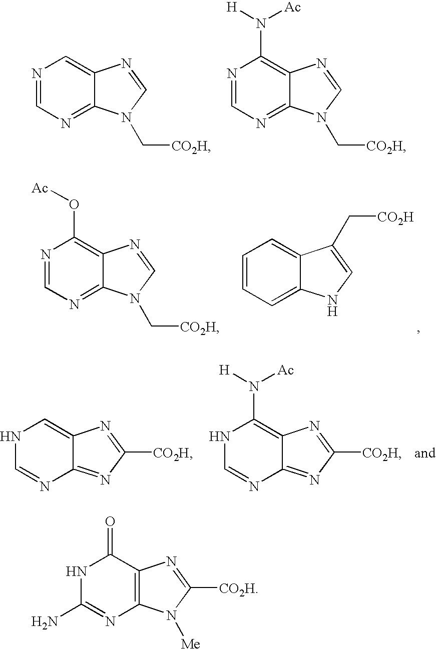

Below is an exemplary compound (IV) of Formula III, for the inhibition of F. tularensis, which includes: 1) a hydroxamic acid as a zinc-binding motif at its terminus, 2) R¹, a dihydroxy phenyl mimic of the first thymine, 3) R², a tryptophan derivative to mimic the adenosine base, and 4) R³, a pyridine derivative to mimic the second thymine base. It is anticipated that those substituents, R¹-R³, will be optimized in a combinatorial manner to maximize the inhibitor binding specificity.

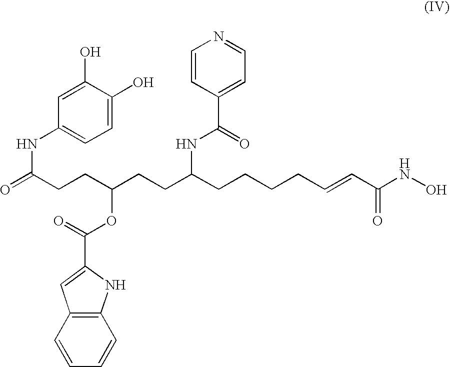

The preparation of structure IV and related derivatives can be accomplished by the route shown below. The known carboxylic acid V (A. Sakamoto, et al. (1987) J. Amer. Chem. Soc. 109:7188) can be converted by standard methods to the alpha-amino acid derivative VI. The R³ substituent can be attached by amidation with an appropriate carboxylic acid derivative to prepare a diverse library of compounds VII bearing a thymine. Ring opening of each lactone with a series of amines bearing the thymine mimic, R¹, can give a library of bisamide derivatives VIII. Acylation of the hydroxyl group in each derivative of VIII with an approariate carboxylic acid dervative bearing the R² substituent designed to mimic adenosine can afford a library of trifunctionalized scaffolds IX. Alkene cross-metathesis can be employed to incorporate the hydroxamic acid or other potential zinc-binding element.

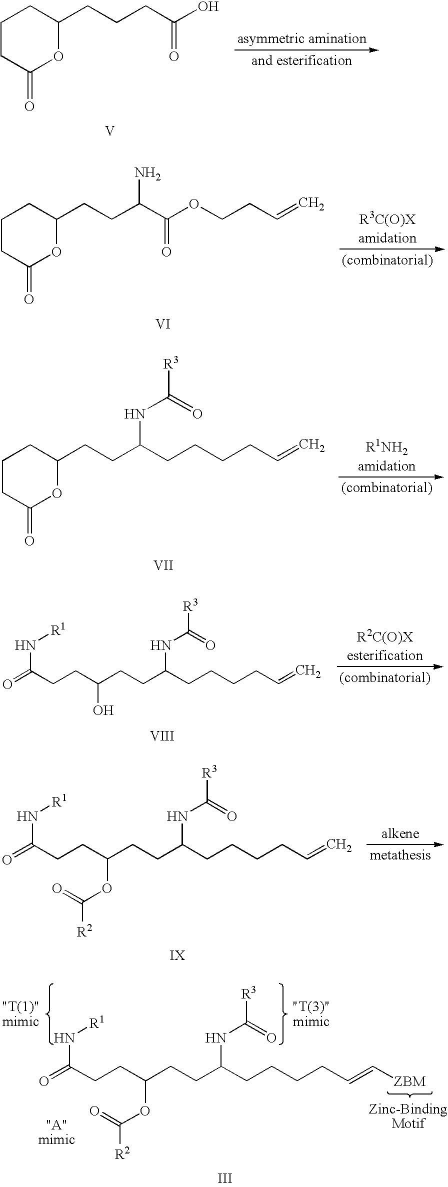

Another route for lead compound identification against the core polymerase domain will be to virtually screen libraries of compounds into potential binding sites on a homology model of the core. The procedure has already been described above for the ZBD domain. The sequence similarity between the core domains of F. tularensis and E. coli is 66%, which is less than the ZBD domains but still very acceptable for accurate homology modeling. Additionally, the core domains of these two bacterial primases have nearly the same number of residues, which simplifies the homology methodology. Preliminary results indicate that an unexpected series of amino acid residues are responsible for the binding of F. tularensis primase to DNA. Further, these residues are in positions and locations that allow for interference by a synthetic or natural inhibitor and by examining the structure for this region. Chemicals or compounds with antimicrobial activity could be generated by rational drug design techniques known in the art. Finally, the process described here will not only apply to antibiotics that can be generated against F. tularensis but will also be applicable for other select infectious agents and organisms that have become resistant to currently existing antibiotics.

The following examples are provided to illustrate various embodiments of the present invention. They are not intended to limit the invention in any way.

### EXAMPLE 1

### Method for Identification of Targets for Development or Selection of Primase Inhibiting Compounds

While the E. coli primase has been well characterized, little or nothing was known of the F. tularensis primase. In separate experiments, the primase of F. tularensis was cloned and placed into an expression vector to make pure protein. To determine whether the F. tularensis primase was active, it was necessary to determine its trinucleotide initiation specificity.

The trinucleotide initiation specificity was predicted by use of a software program which identifies clustering of nucleotide sequences (see U.S. patent application Ser. No. 10/295,030 and Example 4). The software program is capable of predicting the likely trinucleotide binding site of a specific bacterial primase by conducting a mathematical search for clusters of trinucleotides in strings of sequences. This process differs from others which search for overabundant short nucleotide sequences that exist in the genome at a higher frequency than expected. These overabundant sequences are often skewed, that is they have a leading or lagging strand bias. Such an approach has already found that most of the overabundant octanucleotide sequences in the E. coli genome contain the trinucleotide d(CTG) on their leading strand complement. This sequence happens to be the same as the E. coli primase initiation specificity and suggested a link between the two.

Since the contiguous sequence of the F. tularensis genome, which is over 350 contigs, is not available, a method for determining clustering, taking into account window size and threshold, was applied. The method was validated by showing that d(CTG) and its complement d(CAG) are the most clustered trinucleotides in E. coli in windows that varied from 1500 nucleotides to 4500 nucleotides in length. The method also found that d(TAT) and d(ATA) are the most clustered trinucleotides in the genome of F. tularensis and that d(AAT) and d(TTA) were nearly as abundant.

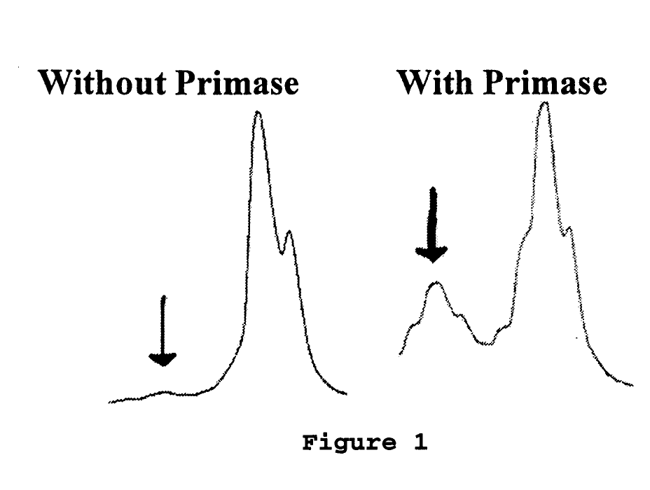

If bacterial chromosome trinucleotide abundance correlates with that bacteria's primase initiation specificity, then the F. tularensis primase specificity was predicted to be either d(TAT), d(ATA), d(AAT), or d(TTA). The standard template sequence into which the variable initiation sequence was placed was d(CACACACACACACAXYZCACACA). Single stranded DNA templates were prepared in which the XYZ portion of the standard sequence was replaced by the desired trinucleotides and separately incubated with primase, the four rNTPs, and magnesium for 1 hour at 30° C. The products were analyzed by DHPLC at 80° C. to separate the primer RNA and template DNA (FIG. 1). The results shown in Table 1 indicate d(TAT) as the primase's initiation specificity.

TABLE 1

| Template Trinucleotide | Primer Yield After 1 Hour |
|---|---|
| d(TAT) | Good |
| d(ATA) | Barely Detected |
| d(TTA) | Barely Detected |
| d(AAA) | Barely Detected |
| d(CTG) | Not Detected |
| d(CAG) | Not Detected |
| d(ATC) | Not Detected |
| d(TAC) | Not Detected |

Since the zinc binding domain (ZBD) is hypothesized to determine the initiation trinucleotide specificity of prokaryotic primases, inhibitors that bind to the initiation specificity determining residues are predicted to block primase activity before the first phosphodiester bond has been made. Alternatively, inhibitors could be made that would prevent the ZBD from binding to DNA. Both routes are expected to inhibit primer synthesis, prevent DNA synthesis from occurring, disrupt bacterial cell division and achieve the desired anti microbial effect. Further, since the specificity determining residues are expected to be unique among certain genus or species of bacteria, the method for inhibitor discovery is expected to provide for generation of narrow spectrum or broad spectrum antibiotics.

Preliminary Model Building of the F. tularensis ZBD Structure

The SYBYL® Composer program (Tripos, Inc.; St. Louis, Mo.) was employed to model the structure of F. tularensis primase based on expected homologies with other primases. Given the high sequence homology and length conservation of the primase ZBD, it was probable that the ZBD structure was highly conserved. After substituting the F. tularensis residues into the available ZBD structure from B. stearothermophilus, SwissProt's energy minimization program was employed to create a model of the ZBD (see “Ftula ZBD” at www.expasy.ch/swissmod/SWISS-MODEL). A comparison of the backbone alpha carbons from this model with the original ZBD structure revealed that no residue was positioned in an unfavorable manner.

Evidence for the Determinants of Initiation Trinucleotide Specificity

The ZBD structure is unique to bacterial primases. Therefore, inhibitors against this domain are hypothesized to be specific for bacteria and perhaps specific for a given bacterial species. The ZBD contains the most conserved sequence of primase's three domains, with the most conserved residues immediately surrounding the zinc binding ligands. The zinc binding residues that have been demonstrated for both E. coli and B. stearothermophilus are Cys40, His43, Cys61, and Cys64 and were expected to be the same in primase from F. tularensis. In the 3D structure of B. stearothemophilus, the zinc stabilizes a zinc ribbon and is bound to residues at the ends of strands 2 and 4. The zinc ribbon is part of a 5-strand antiparallel beta-sheet. The alignment of selected and putative primase gene products was performed (see Table 2, wherein the positions of the predicted trinucleotide specificity residues are indicated by 1, 2, and 3). This alignment resulted in the recognition of both conserved and variable regions that had not previously been thought to play a role in the base specific recognition capability of primases. It is hypothesized that certain regions are important because: 1) among the amino acids that are different between E. coli and F. tularensis, three stood out for their location on an exposed surface while other variable amino acid residues were located in buried helices; 2) the residues were located in a region that contains many hydrophobic and aromatic residues likely to be able to stack against nucleotides in single stranded DNA; and 3) all three of the residues of interest lined up in the same position.

[image]

The “Ftula ZBD” model and the sequence alignment were used to determine whether any of its surface residues were candidates to determine the enzyme's trinucleotide initiation specificity. If the candidate residues were near a potential inhibitor binding site, it should be possible to interfere with the ability of the primase to recognize its trinucleotide and prevent primer synthesis through generation of an inhibitor that bound or interfered with this site. Such inhibitors would be specific for bacteria with the same primase initiation specificity residues, thus providing a method for generating narrow-spectrum antibiotics.

Interestingly, there are only three residues that are both surface exposed and variable in the F. tularensis ZBD: Lys37, Phe51, and Ser67. These residues are on beta strands 2, 3, and 5, respectively, and are aligned across the exposed face of ZBD's beta sheet.

The F. tularensis primase residues Lys37, Phe51, and Ser67 can be separately mutated by site-directed mutagenesis to the ones found in E. coli in an attempt to alter the initiation specificity in a predictable manner. For example, wild-type F. tularensis primase is specific for the trinucleotide d(TAT), but a mutant F. tularensis primase comprising the mutation Lys37His would have a predicted specificity for the trinucleotide d(CAT). Similarly, mutant F. tularensis primases comprising either the mutation Phe51Thr or Ser67His would have a predicted specificity for the trinucleotides d(TTT) or d(TAG), respectively. Each mutant may be prepared as a fusion with glutathione-S-transferase protein, overproduced, and purified in the same way as the wild type protein. The initiation specificity of each mutant may be subjected to a battery of templates that includes not only the predicted specificities but likely alternatives as well. For instance, even though Lys37 is predicted to be responsible for the specificity of the first nucleotide, it is possible that it is responsible for the third nucleotide. Inclusion of the trinucleotide d(TAG) among the test templates may insure this outcome will not be missed.

Preliminary Research to Identify Inhibitor Binding Sites on the ZBD

The SYBYL® SiteID™ program (Tripos) found three potential binding sites on our “Ftula ZBD” model. Briefly, the ZBD surface was covered with water sized spheres, the positional relationship between the spheres determined, and binding sites identified by those spheres that are more than one sphere below the surface. The program identified three potential binding sites/pockets. The binding pockets are identified as:
Pocket 1: Val14, Ala17, Asn57, Ala70, Leu71
Pocket 2: Val22, Tyr26, Val74, Asn88, Leu89
Pocket 3: Cys40, His43, Glu45, Thr47, Ser49

“Pocket 3” was the smallest and could accommodate ten water sized spheres. Pocket 3 is of interest for several reasons: 1) it lies adjacent to the initiation specificity residues described above, 2) it lies to one side of the zinc binding residues Cys40 and His43, and 3) it is composed of very highly conserved residues. Therefore, inhibitors generated to bind to this region and to the adjacent initiation-specificity residues are predicted to be antibiotics with narrow specificity.

The “Ftula ZBD” Pocket 1 was the largest with 15 spheres. It was in the center of the primase ZBD. The pocket coincides with a depression into which a knob from the primase core domain may fit when the two domains interact. The bottom of the depression consists of, clockwise: Val14, Ile10, Leu71, and Val186. The residues surrounding the depression are, clockwise: open space/gap, Lys11, Asn7, Lys3, Val86, Phe82, Thr72, Asp69, and Asn57. Since this site is composed of moderately conserved residues it is predicted that inhibitors directed to this site would have a moderate spectrum of activity.

### EXAMPLE 2

### Thermally Denaturing HPLC Analysis of Primase Activity

Material and methods

Escherichia coli primase was produced and isolated as previously described (Griep, M. A., et al. (1996) Biochemistry 35:8260-8267). Synthetic single-stranded RNA (ssRNA) oligonucleotides with the sequences 5′-AG(UG)₅-3′, 5′-AG(UG)₇-3′, and 5′-AG(UG)₈-3′ were obtained from Invitrogen (Carlsbad, Calif.). Synthetic ssDNA oligonucleotides with the sequences 5′-AG(UG)₅-3′, 5′-AG(UG)₇-3′, 5-AG(UG)₈-3, 5′-AG(TG)₅-3′, 5′-AG(TG)₇-3′, 5′-AG(TG)₈-3′, 5′-(CA)₇CTG(CA)₃-3′, and 5′-CAGA(CA)₅CTG(CA)₃-3′, with and without the 3′ end blocked with a C3 linker, were obtained from the University of Nebraska Medical Center DNA Core Facility. The oligonucleotides were purified on a 20% denaturing polyacrylamide gel electrophoresis (PAGE), visualized by UV shadowing, cut from the gel, and eluted into Tris-EDTA buffer. All oligonucleotides were quantified spectrophotometrically using their respective extinction coefficients. HPLC Buffer A (0.1 M triethylammonium acetate, pH 7.0), Buffer B (0.1 M triethylammonium acetate, 25% acetonitrile v/v), WAVE HPLC Nucleic Acid Fragment Analysis System, and DNASep® HPLC column were from Transgenomic (Omaha, Nebr.). Magnesium acetate, potassium glutamate, Hepes, and DTT were from Sigma (St. Louis, Mo.). Microspin G-25 columns were from Amersham (Piscataway, N.J.). Ribonucleoside triphosphates (rNTPs) and deoxyribonucleoside triphosphates (dNTPs) were from Roche Molecular Biosystems (Mannheim, Germany), (α-³²P]rUTP was from ICN (Costa Mesa, Calif.).

RNA Primer Synthesis

All RNA primer synthesis reactions were performed in 200 μl nuclease-free water containing 50 mM Hepes, 100 mM potassium glutamate, pH 7.5, 10 mM DTT, 10 mM magnesium acetate, and 200 nM ssDNA template. De novo primers were generated by using 3′-blocked ssDNA template, 200 μM rNTPs, and 2 μM primase (FIG. 2A). Inhibition studies of de novo primer synthesis were conducted identically except in the presence of 0, 2.5, 5, 10, 50, or 100 μM inhibitor. Overlong primers were generated by using ssDNA template with a free 3-hydroxyl group, 200 μM rUTP and rGTP, and 200 nM primase (FIG. 2B). All components of the reaction except primase were mixed together and preincubated at 30° C. The reaction was started with the addition of primase (also at 30° C.) and incubated for 1 hour (or as indicated). The concentration of rNTPs and ssDNA and the incubation temperature were used as previously optimized for E. coli primase (Swart and Griep (1995) Biochemistry 34:16097-16106). Control reactions used identical conditions except that the rNTPs or primase was substituted with water. The reactions were stopped by heat inactivation at 65° C. for 10 min, desalted through a Microspin G-25 column, and speed vacuumed to dryness. The pellet was resuspended in 1/10 th the original volume of water.

Thermally Denaturing HPLC (DHPLC) of Oligonucleotides

Eight microliters of the primer synthesis reaction was analyzed by HPLC under thermally denaturing conditions at 80° C. UV detection was performed at 260 nm. A range of buffer gradients was evaluated to determine the optimal conditions for separation of primers. De novo primer synthesis (FIG. 2A) was monitored using a 0.9-ml/min flow rate and a gradient of 0-8.1% acetonitrile over 16 min. The elution profiles of the control RNA and DNA oligonucleotides were also analyzed using a 0.9-ml/min flow rate and a gradient of 0-8.1% acetonitrile over 16 min. Overlong primer synthesis (FIG. 2B) was analyzed with a gradient of acetonitrile from 4.5 to 8.0% over 7 min. Average analysis time was less than 20 minutes per reaction. Data were collected and analyzed in Microsoft® Excel. Fluctuations in retention time caused by variability in time between the injection of the sample and the injection peak were controlled by using the ssDNA template as an internal control relative to which all other peak retention times were measured.

PAGE and Storage Phosphor Autoradiography

De novo primer synthesis was carried out as described above, except that rUTP was substituted with [α-³²P]rUTP. After resuspension of the nucleic acid pellet in loading buffer containing formamide, 3 μl was loaded on a 20% polyacrylamide gel containing 6 M urea and electrophoresed for 14 hours at 300V. The gel was exposed on a storage phosphor screen for 12 hours followed by autoradiography.

Quantitation of RNA Primer Synthesis, Kinetics, and Inhibition

Known amounts of the 16-mer ssRNA 5′-AG (UG)₇-3′ were analyzed by DHPLC. The area under the peak was calculated and a standard curve relating peak area (ΣΔmV*Δt) to picomoles of oligonucleotide was generated. Linear regression yielded the relationship:
P=0.65(±0.05)*A+0.06(±0.09),
where P is pmol 16-mer primer and A is the area of the 16-mer peak calculated from the chromatogram. The R² was 0.98, and the standard error was 0.13. The RNA primers were quantified by comparing the areas under the chromatographic curve to the standard curve. Primer synthesis kinetics data were fit to the equation:
Y=Yₘₐₓ(1−e^{(-kt)}),
where Y is pmol primers synthesized, Yₘₐₓ is the maximum primers synthesized, k is the rate constant, and t is time in seconds.

The concentration of an inhibitor that reduces primase activity by 50% (IC50) was calculated by fitting data to the equation:

$$\% Activity = 100 \% - \frac{100 \% [ I ]}{IC50 + [ I ]}$$

where [I] is the concentration of the inhibitor.
Results

This study determined whether thermally denaturing HPLC was able to measure and differentiate the two modes of in vitro primase activity: de novo and overlong primer synthesis. To measure de novo primer synthesis, primase and rNTPs were used to synthesize RNA primers complementary to a ssDNA template lacking a 3′-hydroxyl group (FIG. 2A). To measure overlong primer synthesis, a template containing a 3′-hydroxyl group was incubated with primase, rUTP, and rGTP in a similar manner yielding an RNA-DNA copolymer (FIG. 2B). Primase products (de novo or overlong primers) were then chromatographically separated from the ssDNA template and analyzed.

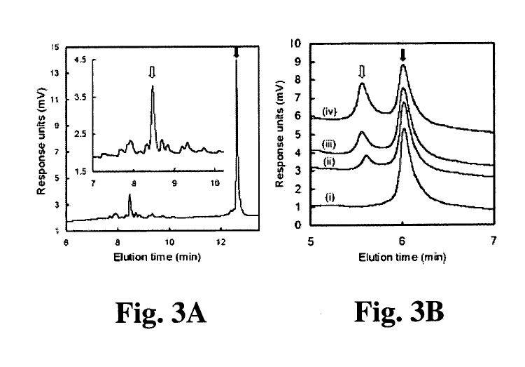

To study de novo primer synthesis (FIG. 2A), primase (2 μM), rNTPs (200 μM), and ssDNA template 5′-CAGA(CA)₅CTG(CA)₃-C3-3′ (200 μM) were incubated together at 30° C. for 1 hour. DHPLC analysis of the de novo primer synthesis reaction yielded a major peak at 8.49±0.01 minutes (FIG. 3A inset, open arrow) surrounded by multiple smaller peaks and a late peak eluting at 12.64 minutes (FIG. 3A, filled arrow). DHPLC analysis of similarly prepared reactions lacking primase generated only one peak that eluted at 12.64 minutes (data not shown), consistent with the late peak being the ssDNA template. Because recombinant primase has been known to copurify with a 3′→5′ exonuclease (Griep and Lokey (1996) Biochemistry 35:8260-8267), control reactions were performed with all reaction components except either rNTPs or primase incubated with ssDNA template overnight. Again, only one peak at 12.64 minutes was observed (data not shown).

To study overlong primer synthesis (FIG. 2B), primase (200 nM), rUTP and rGTP (200 μM each), and ssDNA template 5′-(CA)₇CTG(CA)₃—OH-3′ (200 nM) were incubated together at 30° C. for 1, 2, and 4 hours. DHPLC analysis of the overlong primer synthesis reaction yielded a template peak at 6.05 minutes (FIG. 3B, open arrow) with the overlong RNA-DNA copolymer moiety eluting before the template at 5.40±0.03 minutes (FIG. 3B, filled arrow). The appearance of the early peak was identified as the overlong primer because its production required rGTP and rUTP. In addition, 200 nM primase was necessary for overlong primer synthesis versus 2 μM primase for de novo primer synthesis (data not shown). Despite using a variety of elution gradients or a tetrabutylammonium bromide ion-pairing system to enhance the separation of the “overlong primer” peak from the template peak, it was not possible to obtain baseline separation between the two peaks.

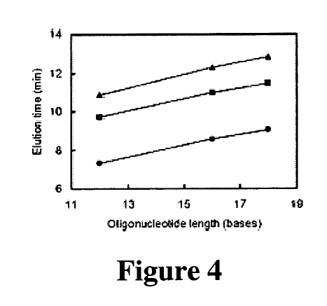

To further interpret the chromatograms, control RNA and DNA oligonucleotides were analyzed: a 12-mer, 5′-r(AG(UG)₅), 5′-d(AG(UG)₅), or 5′-d(AG(TG)₅); a 16-mer, 5′-r(AG(UG)₇), 5′-d(AG(UG)₇), or 5′-d(AG(TG)₇); and an 18-mer, 5′-r(AG(UG)₈), 5′-d(AG(UG)₈), or 5′-d(AG(TG)₈) DHPLC analysis of both the RNA and the DNA control oligonucleotides demonstrated that retention time increased proportionally with respect to oligonucleotide length (FIG. 4). DNA oligonucleotides eluted an average of 3.70 minutes later than their corresponding RNA oligonucleotides, and DNA oligonucleotides that substitute dUMP for dTMP eluted on average 2.42 minutes later than their analogue RNA oligonucleotides. The 16-mer RNA oligonucleotide control eluted at 8.55 min, which was similar to the elution time of 8.49 minutes for the major early peak observed for the primase reaction (FIG. 3A, open arrow), suggesting that the peak was indeed the complementary 16-mer predicted to be synthesized by primase on the ssDNA template.

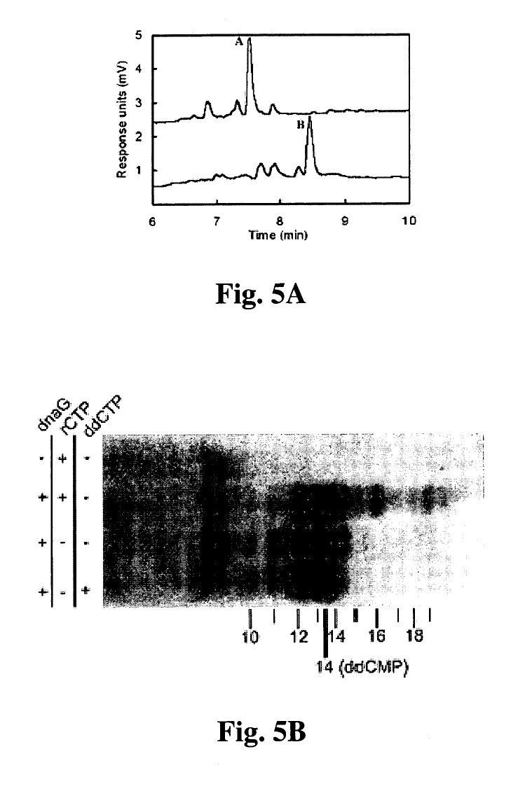

To investigate whether it was possible to examine site-specific nucleotide insertion, the 5′-antepenultimate guanosine in the ssDNA template was exploited by omitting rCTP from the primase reactions. In a de novo primer synthesis reaction lacking rCTP, primase should synthesize a 13-mer primer. DHPLC analysis of the reaction yielded a major peak at 7.52±0.03 minutes with smaller peaks on either side (FIG. 5A, trace A). Extrapolating from the RNA control data (FIG. 4), a 13-mer RNA polymer was predicted to elute at 7.63 min. The observation that primers greater than 13 nucleotides were present in lower abundance suggested that primase was capable of inserting the incorrect basepair at a measurable rate. The same reaction was performed in the presence of 5 μM ddCTP, which was expected to add a ddCMP to the 13-mer RNA oligonucleotide, creating a 14-mer RNA-ddCMP copolymer. DHPLC analysis of the 14-mer yielded a major peak at 8.47±0.01 minutes (FIG. 5A, trace B), which was much later than the predicted 7.92 minutes for an RNA 14-mer. No peaks were observed to be longer than the ddCTP-terminated primer product (FIG. 5A, trace B).

To confirm the HPLC analysis of the site-specific nucleotide insertion, de novo primer synthesis reactions were performed with [α-³²P]UTP, separated via PAGE, and visualized by autoradiography (FIG. 5B). In the absence of primase, no bands were visualized (FIG. 5B, lane 1). In de novo primer synthesis reactions containing all four rNTPs, multiple bands were observed (FIG. 5B, lane 2). Omission of rCTP from the de novo primer synthesis reaction yielded a major band attributed to the 13-mer product, surrounded by several less-intense bands (FIG. 5B, lane 3). Addition of ddCTP to the reaction yielded an intense band that migrated slightly higher than the 13-mer (FIG. 5B, lane 4). Other less intense bands that were of a smaller molecular weight were observed.

While it is difficult to quantitatively measure the sensitivity of the new HPLC assay as compared to radiometric methods, a relative measure can be estimated by FIG. 5A. The HPLC analysis used 8 μl of the de novo primer synthesis reaction, whereas PAGE and storage phosphor autoradiography analysis required 3 μl. Thus, the relative sensitivity of the HPLC analysis of primase activity is approximately 2.5 times less than that of the radiometric method. As previously stated, the average HPLC analysis took 20 min. The radiometric analysis took 14 hours for PAGE at 300V followed by 12 hour to expose the storage phosphor screen.

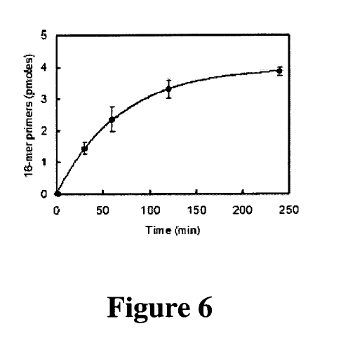

To demonstrate that the HPLC assay can be used quantitatively, the rate of de novo primer synthesis was measured. The peak areas of the 16-mer RNA primers (FIG. 3A, open arrow) from 30-, 60-, 120-, and 240-minute reactions were calculated and converted into picomoles of primers synthesized using a standard curve (see Material and methods). The amount of 16-mer primer synthesized by primase was quantitated at each time point (FIG. 6). The data were fit with the kinetics equation describer hereinabove to yield a Yₘₐₓ of 3.96±0.01 pmol and a rate constant of 0.00251±0.000003 s⁻¹. The R² was 1.00. The previously determined rate constant for primase on a ssDNA template was 0.00083 s⁻¹ (Swart and Griep (1995) Biochemistry 34:16097-16106).

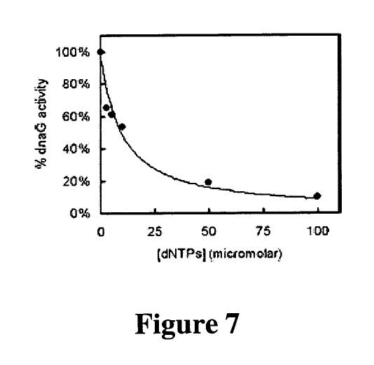

To test the ability of a mixture of dNTPs to inhibit primase activity, de novo primer synthesis was conducted in the presence of 0, 2.5, 5, 10, 50, or 100 μM dNTPs for 1 hour and analyzed by DHPLC. Total primer synthesis was quantitated for each reaction. The amount of primers produced in the absence of dNTPs was set to 100% primase activity with the reduction in primers synthesized reported as a percentage of the uninhibited activity (FIG. 7). The IC50 for dNTPs was determined to be 9.5±1.4 μM. The IC50 value determined previously for d NTPs was 5 μM (Rowen, L., et al. (1978) J. Biol. Chem. 253:770-774).

Discussion

The distinction between primase's two modes of activity (de novo primer synthesis versus elongation from an existing 3′-hydroxyl group) is an important consideration when designing an assay to measure primase activity. The physiologic function of primase is to create de novo primers during DNA replication and not to elongate from the 3′-end of an artificial ssDNA template hairpin. Thus, an assay that is not capable of distinguishing between de novo and overlong primer synthesis generates misleading information, particularly when applied to the characterization of inhibitors. Indeed, a recently described high throughput primase assay which uses synthetic ssDNA templates that were not blocked at their 3′ ends (Zhang, Y., et al. (2002) Anal. Biochem. 304:174-179) therefore measures primarily overlong primer synthesis.

Thermally denaturing HPLC analysis of de novo primer synthesis yielded a major peak that eluted at 8.49 minutes surrounded by smaller peaks (FIG. 3A). Control reactions lacking primase or rNTPs confirmed the peaks that eluted from 7.00 to 10.50 minutes were not degradation products of the ssDNA template. Using control RNA oligonucleotides, it was determined that the major peak observed was indeed the template-length-dependent primer. The smaller peaks surrounding the 16-mer peak probably represented primers that were 16±n nucleotides in length.

Overlong primer synthesis was also observed by DHPLC analysis (FIG. 3B). The major factors contributing to the differences between de novo and overlong primer synthesis were the presence of a C-3′-hydroxyl group on the ssDNA template and the requirement of 10-fold more primase for de novo primer synthesis. This reflected the ability of primase to elongate more efficiently from the existing C-3′-hydroxyl of the DNA primer formed by the hairpin rather than to generate a de novo primer complementary to the 5′-CTG-3′ recognition sequence (Swart and Griep (1995) Biochemistry 34:16097-16106).

This is the first study to compare the elution of RNA and DNA oligonucleotides together on an alkylated nonporous polystyrene-divinylbenzene copolymer microsphere column under thermally denaturing conditions. To interpret the chromatograms of primase activity and to better understand the role that hydrophobicity had on retention time, the differential elution properties of corresponding RNA and DNA oligonucleotides were examined (FIG. 4). The column matrix and ion-pairing buffer used in this study have been reported to separate equivalently sized DNA oligonucleotides based on differences in their sequences (Haefele, R. G., Quality control and purification of oligonucleotides on the WAVE nucleic acid)fragment analysis system, Transgenomic Appl. Note AN103 1-3). The chromatographic separation was based on differential hydrophobicity due to the relatively short alkyl chain of the triethylammoniurn acetate ion-pairing buffer that allows the hydrophobic column matrix to be partially accessible. Thus, equivalent-sized hydrophilic oligonucleotide moieties elute before hydrophobic moieties. Accordingly, the presence of the C-2′-hydroxyl group on an RNA oligonucleotide was predicted to have a shorter elution time than an analogous DNA oligonucleotide.

As expected, retention time was proportional to the size of the oligonucleotide for both RNA and DNA (FIG. 4). However, the loss of the C-2-hydroxyl group between the 5′-rAG(UG)ₙ and the 5′-dAG(UG)ₙ oligonucleotides increased the elution time by 2.42 min, and the gain of the N-5-methyl group in addition to the loss of the C-2-hydroxyl group between the 5′-rAG(UG)ₙ and the 5′-dAG(TG)ₙ oligonucleotides increased the elution time by an average 3.70 min. The differential elution of similar oligonucleotides demonstrated the importance of an oligonucleotide's hydrophobicity on its retention, time at thermally denaturing temperatures.

The contribution that hydrophobicity had on elution time was also demonstrated by the site-specific nucleotide insertion experiments (FIG. 5A). The control RNA oligonucleotide data predicted that a 13-mer and a 14mer RNA oligonucleotide would elute at 7.63 and 7.92 minutes, respectively. The 13-mer primer eluted at 7.52 minutes, which was near its predicted value. In contrast, the 14-mer RNA-ddCMP lacked the hydrophilic C-2′ and C-3′-hydroxyl groups on its terminal nucleotide and eluted at 8.47 minutes, which was much later than the predicted value for a 14-mer RNA oligonucleotide.

While the RNA and DNA oligonucleotides followed the respective predicted elution profiles based on their length, the RNA-DNA copolymer that comprised the overlong primer eluted from the column before the template despite being a longer entity (FIG. 3B). The addition of each C-2′-hydroxyl group was able to decrease the elution time of the RNA-DNA copolymer to a larger extent than each additional nucleotide was able to increase the elution time. The earlier elution of the overlong primer in accord with the data in FIG. 4 indicated that hydrophobicity influenced retention time more than size.

In addition to hydrophobicity and oligonucleotide length, variations in extinction coefficients in short oligonucleotides were accounted for to interpret the chromatograms. Equivalent amounts of two different short oligonucleotides ought to have different peak areas proportional to their extinction coefficients. Thus, quantitation of a particular peak in the chromatogram requires both knowledge of the peak nucleotide content and generation of a standard curve.

The 8.49-minutes 16-mer RNA primer peak was chosen for quantitation because it was the major de novo primer synthesized, and its composition was known. De novo 16-mer primer synthesis for four time points was quantitated using a standard curve (FIG. 6) and the rate constant was calculated. The resulting rate constant of 0.00251 s⁻¹ was nearly three times the rate of 0.00083 s⁻¹ previously reported (Swart and Griep (1995) Biochemistry 34:16097-16106). One explanation for the experimental difference between these two studies is that the previous study used a ssDNA template with an unblocked 3′ end. Thus, the formation of de novo primers and overlong primers occurred simultaneously and competitively in the earlier study, thereby decreasing the observed rate constant for de novo primer synthesis.

The DHPLC assay was also capable of measuring inhibition of primase activity by dNTPs. It has been reported that dNTPs profoundly inhibit the formation of RNA primers by primase (Rowen, L., et al. (1978) J. Biol. Chem. 253 (1978) 770-774). The biological function of this dNTP inhibition may be to limit primase function at the replication fork. This would reduce the length of the RNA primers, cause primase to stall, and provide a deoxyribonucleotide from which the DNA polymerase can elongate. The finding of an IC50 of 9.5 μM (FIG. 7) was comparable to the previously determined IC50 of approximately 5 μM (Rowen, L., et al. (1978) J. Biol. Chem. 253 (1978) 770-774).

The products of the primer synthesis reaction were analyzed by both HPLC and conventional PAGE/autoradiography (FIG. 5). The distribution of peaks observed by HPLC was similar to the banding patterns on the gel. Specifically, the de novo reaction containing all four rNTPs produced at least seven bands (FIG. 5B, lane 2) and the chromatogram also had greater than seven peaks (FIG. 2A). The de novo reaction lacking rCTP yielded a major 13-mer band surrounded by bands of larger and smaller molecular weight (FIG. 5B, lane 3). Likewise, the chromatogram of the same reaction had a major 13-mer peak at 7.52 minutes with smaller peaks on either side (FIG. 5A, trace A). Analysis of the de novo reaction containing ddCTP in place of rCTP by PAGE/autoradiography yielded a major 14-mer band that migrated slightly slower than the 13-mer (FIG. 5B, lane 4) due to its reduced molecular weight as compared to an rCTP containing two more hydroxyl groups. Less intense bands were observed of a smaller molecular weight only. In comparison, the HPLC analysis of the same reaction yielded a major 14-mer peak at 8.47 minutes with no peaks observed at later elution times (FIG. 5A, trace B). While the banding pattern aligned well with the HPLC chromatogram, the intensities of the bands did not correlate linearly with the peak areas. Presumably this is due to two factors: (1) differences in extinction coefficients of the short RNA primers and (2) various size primers able to incorporate differing amounts of [α-³²P] UTP.

In conclusion, thermally denaturing HPLC analysis of primase activity was capable of reproducing known properties of primase including de novo or overlong primer synthesis. DHPLC analysis yielded quantitative information on the size of the primers synthesized and provided a method to screen and determine the IC50 for a direct inhibitor of primase. DHPLC analysis was found to be more rapid than the radiometric assays of primase. Further, the DHPLC assay is automated and scalable for high-throughput analysis while providing critical information about the size and quantity of primers produced.

### EXAMPLE 3

### Staphylococcus aureus Primase

Cloning of S. aureus Primase

The dnaG gene from S. aureus was identified in GenBank and primers SAdnaGF 5′-CATGCCATGGGGAGATTTAATTTGCGAATAGATC-3′ and SAdnaGR 5′-GGAATTCAAATCACATGCTACATGCGTTC-3′ were used to amplify the dnaG gene product from S. aureus ATCC 29213 and insert restriction sites (underlined) into the amplicon. The PCR product was digested and inserted into a similarly prepared pET41-A vector (Novagen; Madison, Wisc.) and transformed into E. coli DH5a cells. Sequencing was employed to verify the insert. The plasmid pET41-A SA dnaG was then transformed into E. coli BL21 cells.

Primase Protein Production and Purification

E. coli BL21 cells containing the primase clone were grown in 2YT media with kanamycin in overnight cultures to an OD600 of 1.0. The cells were then induced with 0.5 mM isopropyl-beta-D-thiogalactopyranoside (IPTG) for 2 hours at 30° C. The cells were then lysed with lysozyme into 50 mM Tris, 5 mM EDTA. Primase was purified on a Sepharose 4B-glutathione column followed by ion-exchange chromatography.

Data Analysis

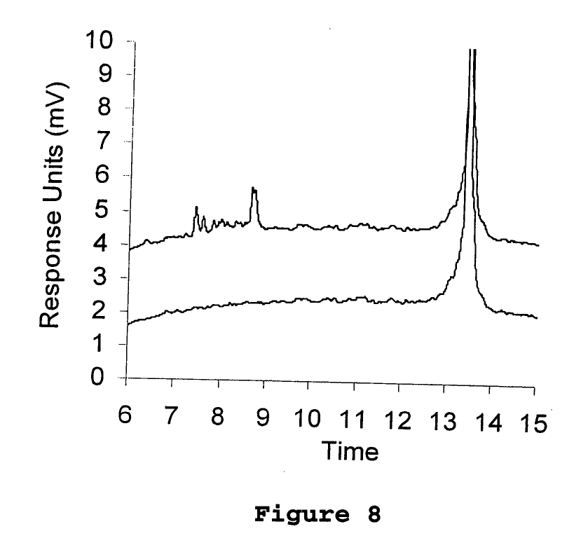

To determine the binding specificity of S. aureus, the purified protein was incubated with 16 different ssDNA templates of the sequence 5′-(CA)₇XYZ(CA)₃-3′, where XYZ is TAT, ATA, TTA, AAT, CAT, TTT, TAG, CTG, CAG, CTT, GAA, AAG, TTC, AAA, TAA, and ATT, under conditions described in Example 2. Only the template where XYZ=TTA demonstrated primase activity (FIG. 8).

### EXAMPLE 4

### Identification of Trinucleotide Clustering

The lagging strand in DNA replication has to replicate its complement in the 5′-3′ direction. In bacteria, this is done by the construction of relatively short fragments, known as the Okazaki fragments which are constructed in the 5′-3′ direction and then ligated (Ogawa and Okazaki (1980) Annual Rev. Biochem. 49:421-457). The production of an Okazaki fragment is initiated by the binding of primase to a recognition site. In E. coli the recognition site is known to contain the triplet CTG (Hiasa, H., et al. (1989) Gene, 84:9-16).

It appears that the binding of primase to its recognition site is a stochastic process. The existence of multiple recognition sites in the neighborhood would increase the probability that binding would occur. Therefore it is hypothesized that there is an evolutionary pressure for the clustering of these recognition sequences in the appropriate regions. Clearly this tendency would be modulated by having to contend with other evolutionary pressures.

Clustering can be defined as follows. Let Wᵥ(k) be a window of length v, defined such that:

$${W}_{v} ( k ) = \{ \begin{matrix}1 & 1 \leq k \leq v \\ 0 & otherwise\end{matrix}$$

Let χ_{X}(n) be an indicator function which is one when the n^{th} triplet is the codon X and zero otherwise. A cluster of codon X exists in the interval [m,m+v] when

$${\sum }_{k = 1}^{N} {χ}_{X} ( m + k ) {W}_{v} ( k ) > τ$$

where τ is an experimentally determined threshold. The number of clusters in the genome is counted for a particular codon. The relative level of clustering is then obtained by comparing the value for a particular cluster against the number of clusters of other codons. However, there is a dependence of the number of clusters on the window size and threshold. In order to incorporate the effect of the window size and threshold on the observation, a relative clustering parameter can be defined as rcp_{X}(v,τ). Let K_{X}(v,τ) be the number of clusters of the codon X in the genome for a given window size v and threshold τ. Define T(v,τ) to be the total number of clusters of all codons for window size v and threshold τ. The relative clustering parameter is defined as

$${rcp}_{X} ( v , τ ) = \frac{{K}_{X} ( v , τ )}{T ( v , τ )}$$

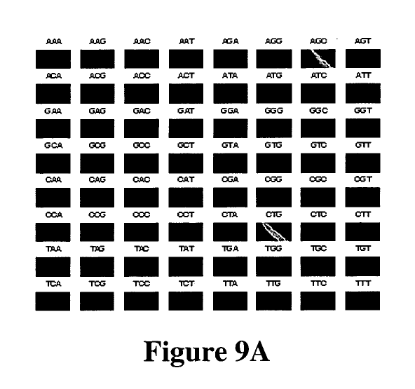

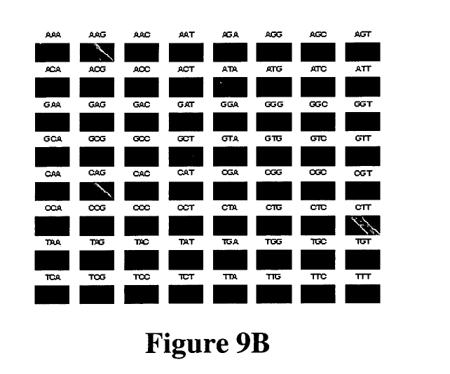

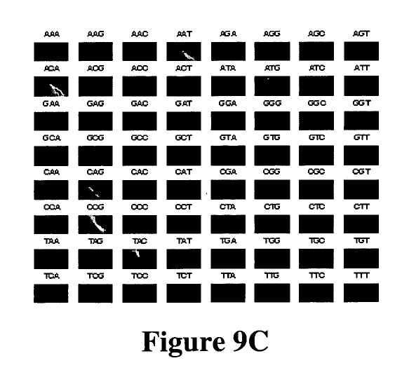

In order to visualize the relative clustering parameter (RCP) for different window sizes and threshold data, the RCP value may be converted to a color which can be displayed as a function of window size and threshold. An example of such a display for E. coli is shown in FIG. 9A. It can be seen from FIG. 9A that there are two sets of triplets which shown a high degree of clustering, namely CTG and AGC. Notably, CTG is the primase binding site. FIGS. 9B and 9C show the clustering for B. anthracis and Y. pestis, respectively. The trinucleotides which show a high level of clustering are identified as candidates for further analysis as possible binding sites for primase.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for identifying the initiation sequence of a bacterial primase comprising:
a) contacting said bacterial primase with a template nucleic acid molecule comprising a candidate initiation sequence;
b) placing the mixture of step a) under conditions suitable for primase activity; and
c) performing thermally denaturing high performance liquid chromatography on the products of step b);
wherein the presence of a nucleic acid molecule other than said template nucleic acid molecule indicates that said candidate initiation sequence is said initiation sequence of said bacterial primase.

2. The method of claim 1, wherein said nucleic acid molecule other than said template nucleic acid molecule is an RNA primer.

3. The method of claim 1, wherein said template nucleic acid molecule is single-stranded DNA.

4. The method of claim 3, wherein said single-stranded DNA is blocked at the 3′ end.

5. The method of claim 1, wherein said bacteria is selected from the group consisting of: Staphylococci, S. aureus, Streptococci, S. pneumoniae, Clostridia, C. perfringens, C. tetani, Neisseria, N. gonorrhoea, Enterobacteriaceae, Helicobacter, H. pylori, Vibrio, V. cholerae, Capylobacter, C. jejuni, Pseudomonas, P. aeruginosa, Haemophilus, H. influenzae, Bordetella, B. pertussis, Mycoplasma, M. pneumoniae, Ureaplasma, U. urealyticum, Legionella, L. pneumophila, Treponema, Leptospira, Borrelia, B. burgdorferi, Mycobacteria, M. tuberculosis, M. smegmatis, Listeria, L. monocytogenes, Actinomyces, A. israelii, Nocardia, N. asteroides, Chlamydia, C. trachomatis, Rickettsia, Coxiella, Rochalimaea, Brucella, Yersinia, Y. pestis, Francisella, F. tularensis, Bacillus, B. anthracis, B. subtilis, and Pasteurella.

6. The method of claim 5, wherein said bacteria is selected from the group consisting of: F. tularensis, S. aureus, B. anthracis, H. pylori, M. tuberculosis, and Y. pestis.

7. The method of claim 6, wherein said bacteria is F. tularensis.

8. The method of claim 1, wherein said candidate initiation sequence is identified by searching for the presence of trinucleotides present in the bacterial genome at a high clustering frequency.

9. The method of claim 8, wherein said search employs an algorithm which accounts for window size and threshold data.

10. A method for identifying a compound which inhibits bacterial primase activity comprising:
a) contacting said bacterial primase with a template nucleic acid molecule comprising the initiation sequence of said bacterial primase and a test compound;
b) placing the mixture of step a) under conditions which promote primase activity; and
c) performing thermally denaturing high performance liquid chromatography on the products of step b);
wherein the detection of a nucleic acid molecule other than said template nucleic acid molecule, in the absence but not the presence of said compound, indicates said compound inhibits bacterial primase activity.

11. The method of claim 10, wherein said bacteria is selected from the group consisting of: Staphylococci, S. aureus, Streptococci, S. pneumoniae, Clostridia, C. perfringens, C. tetani, Neisseria, N. gonorrhoea, Enterobacteriaceae, E. coli, Helicobacter, H. pylori, Vibrio, V. cholerae, Capylobacter, C. jejuni, Pseudomonas, P. aeruginosa, Haemophilus, H. influenzae, Bordetella, B. pertussis, Mycoplasma, M. pneumoniae, Ureaplasma, U. urealyticum, Legionella, L. pneumophila, Treponema, Leptospira, Borrelia, B. burgdorferi, Mycobacteria, M. tuberculosis, M. smegmatis, Listeria, L. monocytogenes, Actinomyces, A. israelii, Nocardia, N. asteroides, Chlamydia, C. trachomatis, Rickettsia, Coxiella, Rochalimaea, Brucella, Yersinia, Y. pestis, Francisella, F. tularensis, Bacillus, B. anthracis, B. subtilis, B. stearothermophilus, and Pasteurella.

12. The method of claim 11, wherein said bacteria is selected from the group consisting of: F. tularensis, S. aureus, B. anthracis, H. pylori, M. tuberculosis, and Y. pestis.

13. The method of claim 12, wherein said bacteria is F. tularensis.

14. A method for identifying a compound which inhibits bacterial primase activity comprising:
a) obtaining a computer model of the zinc-binding domain of said bacterial primase;
b) identifying amino acids of said bacterial primase which are heterologous to the corresponding amino acids of at least one other bacterial primase, said other bacterial primase recognizing a trinucleotide initiation site different than the initiation site recognized by the bacterial primase of step a); and
c) identifying likely compound binding sites on said computer model of step a);
wherein said compound is an inhibitor of bacterial primase activity if said compound binding sites on the primase of step c) co-localize with the heterologous amino acids of step b).

15. The method of claim 14, wherein the compound is further characterized by DHPLC.

16. The method of claim 14, wherein the compound is further characterized by incubation with the bacteria expressing said bacterial primase.

17. The method of claim 14, wherein the heterologous amino acids of step b) determine the initiation specificity of said bacterial primer.

18. The method of claim 17, wherein the initiation specificity determining amino acids are further characterized by site-directed mutagenesis.

19. The method of claim 14, wherein said bacteria is selected from the group consisting of: Staphylococci, S. aureus, Streptococci, S. pneumoniae, Clostridia, C. perfringens, C. tetani, Neisseria, N. gonorrhoea, Enterobacteriaceae, E. coli, Helicobacter, H. pylori, Vibrio, V. cholerae, Capylobacter, C. jejuni, Pseudomonas, P. aeruginosa, Haemophilus, H. influenzae, Bordetella, B. pertussis, Mycoplasma, M. pneumoniae, Ureaplasma, U. urealyticum, Legionella, L. pneumophila, Treponema, Leptospira, Borrelia, B. burgdorferi, Mycobacteria, M. tuberculosis, M. smegmatis, Listeria, L. monocytogenes, Actinomyces, A. israelii, Nocardia, N. asteroides, Chlamydia, C. trachomatis, Rickettsia, Coxiella, Rochalimaea, Brucella, Yersinia, Y. pestis, Francisella, F. tularensis, Bacillus, B. anthracis, B. subtilis, B. stearothermophilus, and Pasteurella.

20. The method of claim 19, wherein said bacteria is selected from the group consisting of: F. tularensis, S. aureus, B. anthracis, H. pylori, M. tuberculosis, and Y. pestis.

21. The method of claim 20, wherein said bacteria is F. tularensis.

22. A kit for performing the method of claim 10 comprising:
a) a set of single-stranded DNA molecules, each with a different trinucleotide comprising G. A, C, and T nucleotides and each being capable of binding a primase;
b) primase buffers;
c) ribonucleoside triphosphates (rNTPs); and
d) a magnesium salt.

23. The kit of claim 22, further comprising at least one element selected from the group consisting of:
a) an HPLC column;
b) wash buffers;
b) elution buffers; and
d) instruction material.

24. A compound which inhibits the activity of a bacterial primase, said compound having a formula selected from the group consisting of: 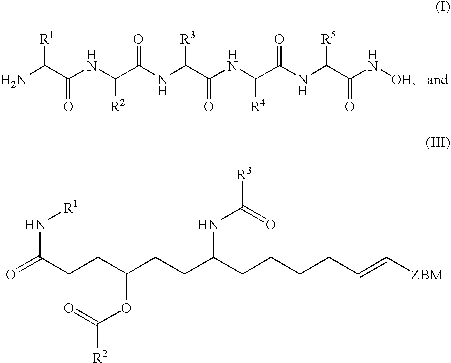
wherein substituents R¹, R², and R³ mimic the three nucleotides of the initiation site of said bacterial primase, wherein substituents R⁴ and R⁵ of Formula I are H or are substituents which increase the binding specificity of the compound for the primase, and wherein ZBM is a zinc-binding motif.

25. The compound of claim 24, wherein the compound is of the formula:

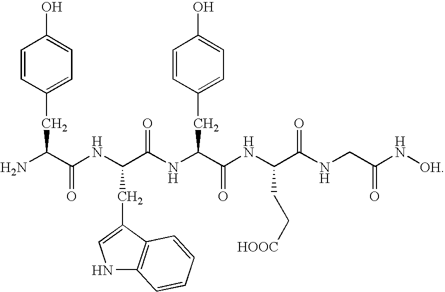

26. The compound of claim 24, wherein the compound is of the formula:

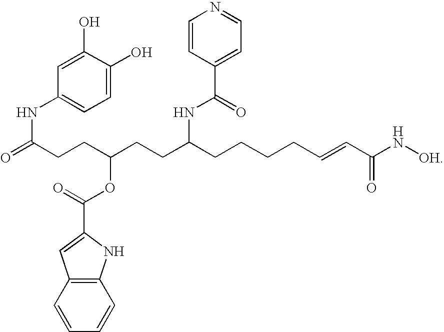

27. The compound of claim 24, wherein said bacteria is selected from the group consisting of: Staphylococci, S. aureus, Streptococci, S. pneumoniae, Clostridia, C. perfringens, C. tetani, Neisseria, N. gonorrhoea, Enterobacteriaceae, E. coli, Helicobacter, H. pylori, Vibrio, V. cholerae, Capylobacter, C. jejuni, Pseudomonas, P. aeruginosa, Haemophilus, H. influenzae, Bordetella, B. pertussis, Mycoplasma, M. pneumoniae, Ureaplasma, U. urealyticum, Legionella, L. pneumophila, Treponema, Leptospira, Borrelia, B. burgdorferi, Mycobacteria, M. tuberculosis, M. smegmatis, Listeria, L. monocytogenes, Actinomiyces, A. israelii, Nocardia, N. asteroides, Chlamydia, C. trachomatis, Rickettsia, Coxiella, Rochalimaea, Brucella, Yersinia, Y. pestis, Francisella, F. tularensis, Bacillus, B. anthracis, B. subtilis, B. stearothermophilus, and Pasteurella.

28. The compound of claim 27, wherein said bacteria is selected from the group consisting of: F. tularensis, S. aureus, B. anthracis, H. pylori, M. tuberculosis, and Y. pestis.

29. The compound of claim 28, wherein said bacteria is F. tularensis.


SEQUENCE LISTING (36 sequences)

SEQ ID NO: 1 - DNA, 23 bp, Artificial Sequence
  Synthetic Sequence
cagacacacacacactgcacaca

SEQ ID NO: 2 - RNA, 16 nt, Artificial Sequence
  Synthetic Sequence
agugugugugugugug

SEQ ID NO: 3 - protein, 5 aa, Artificial Sequence
  Synthetic Sequence
YWYEX

SEQ ID NO: 4 - DNA, 23 bp, Artificial Sequence
  Synthetic Sequence
cacacacacacacannncacaca

SEQ ID NO: 5 - protein, 113 aa, Yersinia pestis Taxon 532
MAGRIPRVFINDLLARTDIIDLIDARVKLKKQGKNYHACCPFHHEKTPSFTVNGEKQFYHCFGCGAHGNAVDFLMNYDRLEFVESIEELATMHGLEVPYEAGSGTTQIERHQR

SEQ ID NO: 6 - protein, 112 aa, Streptococcus pneumoniae
MEVLCMVDKQVIEEIKNNANIVEVIGDVISLQKAGRNYLGLCPFHGEKTPSFSVVEDKQFYHCFGCGRSGDVFKFIEEYQGVTFMEAVQILGQRVGIEVEKPLYSEQKPASP

SEQ ID NO: 7 - protein, 111 aa, s aureus dnaG
MRIDQSIINEIKDKTDILDLVSEYVKLEKRGRNYIGLCPFHDEKTPSFTVSEDKQICHCFGCKKGGNVFQFTQEIKDISFVEAVKELGDRVNVAVDIEATQSNSNVQIASD

SEQ ID NO: 8 - protein, 113 aa, Pseudomonas aeruginosa
MAGLIPQSFIDDLLNRTDIVEVVSSRIQLKKTGKNYSACCPFHKEKTPSFTVSPDKQFYYCFGCGAGGNALGFVMDHDQLEFPQAVEELAKRAGMDVPREERGGRGHTPRQPT

SEQ ID NO: 9 - protein, 109 aa, Mycoplasma pneumoniae
MTSPTSLDQLKQQIKIAPIVEHYAIKLKKKGKDFVALCPFHADQNPSMTVSVAKNIFKCFSCQVGGDGIAFIQKIDQVDWKTALNKALSILNLDSQYAVNFYLKEVDPK

SEQ ID NO: 10 - protein, 113 aa, Mycobacterium tuberculosis CDC1
MSGRISDRDIAAIREGARIEDVVGDYVQLRRAGADSLKGLCPFHNEKSPSFHVRPNHGHFHCFGCGEGGDVYAFIQKIEHVSFVEAVELLADRIGHTISYTGAATSVQRDRGS

SEQ ID NO: 11 - protein, 115 aa, Listeria monocytogenes
MARIPEEVIDQVRNQADIVDIIGNYVQLKKQGRNYSGLCPFHGEKTPSFSVSPEKQIFHCFGCGKGGNVFSFLMEHDGLTFVESVKKVADMSHLDVAIELPEERDTSNLPKETSE

SEQ ID NO: 12 - protein, 112 aa, Francisella tularnesis
MAKKVSNSFIKELVATADIVDVVSRYVNLKKTGKNYKGCCPFHNEKTPSFFVNPEKNFYHCFGCQASGDALTFVKNINKLEFIDAVKNLAEIVGKPVEYENYSQEDIQKEQL

SEQ ID NO: 13 - protein, 113 aa, E. coli K12 Primase
MAGRIPRVFINDLLARTDIVDLIDARVKLKKQGKNFHACCPFHNEKTPSFTVNGEKQFYHCFGCGAHGNAIDFLMNYDKLEFVETVEELAAMHNLEVPFEAGSGPSQIERHQR

SEQ ID NO: 14 - protein, 106 aa, Clostridium tetani
MISKDVIQKVKESNDILDVISERVRLKRSGRYYMGLCPFHNEKSPSFTVTPNKQIYKCFGCGEAGNVITFVMKTRNLPFVDAVHLLADRANIEVTYENGEAPKKDA

SEQ ID NO: 15 - protein, 108 aa, Clostridium perfringens
MRISEEIIEKVKEQNDIVDVVSDVVRLKRAGRNFSGLCPFHNEKSPSFSVSPDKQIFKCFGCGEAGNVISFVMKTKNLNFVDAVKELADRANIIIPIEDGKQSESQKK

SEQ ID NO: 16 - protein, 103 aa, Campylobacter jejuni
MITKESIENLSQRLNIVDIIENYIEVKKQGSSFVCICPFHADKNPSMHINPIKGFYHCFACKAGGDAFKFVMDYEKLSFADAVEKVASLSNFTLSYTKEKQEN

SEQ ID NO: 17 - protein, 109 aa, Borrelia burgdorferi
MKYLQTVASMKSKFDIVAIVEQYIKLVKSGSAYKGLCPFHAEKTPSFFVNPLQGYFYCFGCKKGGDVIGFLMDMEKINYNDALKILCEKSGIHYDDLKISRGSENKNEN

SEQ ID NO: 18 - protein, 113 aa, Bacillus subtilis
MGNRIPDEIVDQVQKSADIVEVIGDYVQLKKQGRNYFGLCPFHGESTPSFSVSPDKQIFHCFGCGAGGNVFSFLRQMEGYSFAESVSHLADKYQIDFPDDITVHSGARPESSG

SEQ ID NO: 19 - protein, 114 aa, Bacillus stearothermophilus
MGHRIPEETIEAIRRGVDIVDVIGEYVQLKRQGRNYFGLCPFHGEKTPSFSVSPEKQIFHCFGCGAGGNAFTFLMDIEGIPFVEAAKRLAAKAGVDLSVYELDVRGRDDGQTDE

SEQ ID NO: 20 - protein, 113 aa, B antrhacis dnaG
MGNRIPEEVVEQIRTSSDIVEVIGEYVQLRKQGRNYFGLCPFHGENSPSFSVSSDKQIFHCFGCGEGGNVFSFLMKMEGLAFTEAVQKLGERNGIAVAEYTSGQGQQEDISDD

SEQ ID NO: 21 - RNA, 12 nt, Artificial Sequence
  Synthetic sequence
agugugugugug

SEQ ID NO: 22 - RNA, 18 nt, Artificial Sequence
  Synthetic sequence
agugugugugugugugug

SEQ ID NO: 23 - DNA, 12 bp, Artificial Sequence
  Synthetic sequence
agtgtgtgtgtg

SEQ ID NO: 24 - DNA, 16 bp, Artificial Sequence
  Synthetic sequence
agtgtgtgtgtgtgtg

SEQ ID NO: 25 - DNA, 18 bp, Artificial Sequence
  Synthetic sequence
agtgtgtgtgtgtgtgtg

SEQ ID NO: 26 - DNA, 23 bp, Artificial Sequence
  Synthetic sequence
cacacacacacacactgcacaca

SEQ ID NO: 27 - DNA, 23 bp, Artificial Sequence
  Synthetic sequence
cagacacacacacactgcacaca

SEQ ID NO: 28 - DNA, 34 bp, Artificial Sequence
  Synthetic sequence
catgccatggggagatttaatttgcgaatagatc

SEQ ID NO: 29 - DNA, 29 bp, Artificial Sequence
  Synthetic sequence
ggaattcaaatcacatgctacatgcgttc

SEQ ID NO: 30 - DNA, 23 bp, Artificial Sequence
  Synthetic Sequence
cacacacacacacannncacaca

SEQ ID NO: 31 - RNA, 16 nt, Artificial Sequence
  Synthetic sequence
gucuguguguguguga

SEQ ID NO: 32 - DNA, 38 bp, Artificial Sequence
  Synthetic sequence
cacacacacacacactgcacacagugugugugugugug

SEQ ID NO: 33 - DNA, 6 bp, Artificial Sequence
  Synthetic Sequence
augcgu

SEQ ID NO: 34 - DNA, 16 bp, Artificial Sequence
  Synthetic Sequence
agugugugugugugug

SEQ ID NO: 35 - DNA, 12 bp, Artificial Sequence
  Synthetic Sequence
agugugugugug

SEQ ID NO: 36 - DNA, 18 bp, Artificial Sequence
  Synthetic Sequence
agugugugugugugugug